(19) United States
(12) Patent Application Publication
(10) Pub. No.: US 2019/0380967 A1
(43) Pub. Date: Dec. 19, 2019

(54) PREPARATION OF MICROPARTICLES OF AN ACTIVE INGREDIENT

(71) Applicant: Savior Lifetec Corporation, Miaoli County (TW)

(72) Inventors: Amy LIU, Miaoli County (TW); Alex CHOU, Miaoli County (TW); Shih-Hsie PAN, Princeton, NJ (US); Mannching Sherry KU, Thiells, NY (US)

(73) Assignee: Savior Lifetec Corporation, Miaoli County (TW)

(21) Appl. No.: 16/479,219

(22) PCT Filed: Jan. 23, 2018
(86) PCT No.: PCT/US18/14763

§ 371 Date: Jul. 19, 2019

Related U.S. Application Data
(60) Provisional application No. 62/449,566, filed on Jan. 23, 2017.

(51) Int. Cl. A61K 9/16; A61K 31/519; A61K 38/26; A61K 38/09

(52) U.S. Cl. CPC A61K 9/1694; A61K 31/519; A61K 9/1647; A61K 38/09; A61K 38/26

(57) ABSTRACT

Disclosed herein is a method for producing microparticles of an active ingredient via an in-line recirculating mixing system, wherein the in-line recirculating mixing system comprises a mixer and a conduit couple to the mixer. The method disclosed herein comprises the steps of: (a) forming a continuous phase of a medium in the in-line recirculating mixing system; (b) allowing the continuous phase of the medium of the step (a) to come into contact with a first mixture of the active ingredient, a polymer and a solvent at a site in the conduit, thereby forming a second mixture, in the conduit; and (c) allowing the second mixture of the step (b) to enter the mixer and circulate in the in-line recirculating mixing system until the microparticles of the active ingredient are formed.

### CROSS-REFERENCES TO OTHER RELATED APPLICATION

This application is a U.S. National Stage Filing under 35 U.S.C. 371 from International Patent Application Serial No. PCT/US2018/014763, filed Jan. 23, 2018, and published on Jul. 26, 2018, which claims benefit to U.S. Provisional application No. 62/449,566 filed Jan. 23, 2017, the disclosure of which is incorporated herein by reference.

### BACKGROUND OF THE INVENTION

### 1. Field of the Invention

The present disclosure relates to the manufacture of microparticles; more particularly, to the manufacture of microparticles of an active ingredient(s) using an in-line recirculating mixing system.

### 2. Description of Related Art

In recent years, intensive research is drawn to the development of microparticles with controlled-release properties. These microparticles may release the active ingredient(s) over a long period of time, from days to months, in a controlled manner, thereby rendering polymeric microparticles the mainstream therapeutics.

While it is essential to consider the particle size distribution, drug load, and drug release profile of the microparticles when designing microparticles; nevertheless, the yield of the producing process is equally crucial. However, the most adopted process for producing polymeric microparticles, such as the method taught by U.S. Pat. No. 6,534,094, fails to produce microparticles having a uniform distribution of the particle size, resulting in subsequent sieving for the production of microparticles with desirable sizes. The sieving inevitably lowers the production yield of the afore-mentioned method

In view of the above, there exists in this art a need of an improved method for producing microparticles that address the afore-mentioned problems or insufficiency in the existing art.

### SUMMARY

The following description presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a method for producing microparticles of an active ingredient(s) using an in-line recirculating mixing system. The method disclosed herein gives rise to microparticles having a narrow distribution in particle size, and an improved yield of production. The in-line recirculating mixing system comprises a mixer, and a conduit coupled to the mixer, thereby forming a closed mixing system. The present method comprises the steps of:

(a) forming a continuous phase of a medium in the in-line recirculating mixing system;

(b) allowing the continuous phase of the medium of the step (a) to come into contact with a first mixture of the active ingredient(s), a polymer and a solvent at a site in the conduit, thereby forming a second mixture, in the conduit; and

(c) allowing the second mixture of the step (b) to enter the mixer and circulate in the in-line recirculating mixing system until the microparticles of the active ingredient(s) are formed.

According one embodiment of present invention, the first mixture and the medium are mixed in a volume ratio of about 1:20 to 1:1,200.

Examples of the active ingredient(s) include, but are not limited to, a physiologically active peptide, an antitumor agent, an antibiotic, an antipyretic agent, an analgesic, an anti-inflammatory agent, an anti-tussive expectorant, a sedative, a muscle relaxant, an antiepileptic, an antiulcer agent, an anti-depressant, an anti-allergic agent, a cardiotonic, an anti-arrhythmic agent, a vasodilator, a hypotensive diuretic, an antidiabetic, an anti-hyperlipidemic agent, an anti-coagulant, a hemolytic, an anti-tuberculosis agent, a hormone, a narcotic antagonist, a bone resorption suppressor, a osteogenesis promoter and an angiogenesis inhibitor.

According to embodiments of the present disclosure, the physiologically active peptides may be selected from the group consisting of, growth hormone releasing peptide (GHRP), luteinizing hormone-releasing hormone (LHRH), somatostatin, bombesin, gastrin releasing peptide (GRP), calcitonin, bradykinin, galanin, melanocyte stimulating hormone (MSH), growth hormone releasing factor (GRF), amylin, tachykinins, secretin, parathyroid hormone (PTH), enkephalin, endothelin, calcitonin gene releasing peptide (CGRP), neuromedins, parathyroid hormone related protein (PTHrP), glucagon, neurotensin, peptide YY (PYY), glucagon-like peptide-1 (GLP1), liraglutide, exenatide, lixisenatide, albiglutide, dulaglutide, taspoglutide, semaglutide, vasoactive intestinal peptide (VIP), pituitary adenylate cyclase activating peptide (PACAP), motilin, substance P, neuropeptide Y (NPY), thyroid stimulating hormone (TSH), insulin, somatostatin, somatostatin derivative, growth hormones, prolactin, adrenocorticotropic hormone (ACTH), ACTH derivatives (e.g., ebiratide), thyrotropin-releasing hormone and salts and derivatives thereof, luteinizing hormone (LH), follicle-stimulating hormone (FSH), vasopressin, vasopressin derivative, oxytocin, calcitonin, gastrin, pancreozymin, cholecystokinin, angiotensin, human placental lactogen, human chorionic gonadotropin (HCG), enkephalin, enkephalin derivatives, endorphin, kyotorphin, interferons, interleukins, tuftsin, thymopoietin, thymosin, thymostimulin, thymic humoral factor (THF), blood thymic factor (FTS) and derivative thereof, other thymic factors, tumor necrosis factor (TNF), colony-stimulating factors (e.g., CSF, GCSF, GMCSF, MCSF), dynorphin, caerulein, bradykinin, urokinase, asparaginase, kallikrein, insulin-like growth factors (IGF-I, IGF-II), nerve growth factor (NGF), cell growth factors (e.g., EGF, TGF-.alpha., TGF-.beta., PDGF, acidic FGF, basic FGF), bone morphogenic factor (BMP), nerve nutrition factors (e.g., NT-3, NT-4, CNTF, GDNF, BDNF), blood coagulation factors VIII and IX, lysozyme chloride, polymixin B, colistin, gramicidin, bacitracin, erythropoietin (EPO), thrombopoietin (TPO), and endothelin-antagonistic peptides, and analogs and fragments thereof. In one preferred embodiment, the active ingredient(s) is LH-RH or an analog thereof, still more preferably leuprorelin or leuprorelin acetate. In one preferred embodiment, the active ingredient(s) is GLP-1 or an analog thereof, still more preferably exenatide.

Examples of the antitumor agents include, but are not limited to, bleomycin, methotrexate, actinomycin D, mitomycin C, binblastin sulfate, bincrystin sulfate, daunorubicin, adriamycin, neocarzinostatin, cytosinearabinoside, fluorouracil, tetrahydrofuryl-5-fluorouracil, krestin, picibanil, lentinan, levamisole, bestatin, azimexon, glycyrrhizin, polyl:C, polyA:U and polyICLC.

Examples of the antibiotics include, but are not limited to, gentamicin, dibekacin, Kanendomycin, lividomycin, tobramycin, amikacin, fradiomycin, sisomycin, tetracycline hydrochloride, oxytetracycline hydrochloride, rolitetracycline, doxycycline hydrochloride, ampicillin, piperacillin, ticarcillin, cefalothin, cefaloridine, cefotiam, cefsulodin, cefmenoxime, cefmetazole, cefazolin, cefotaxime, cefoperazon, ceftizoxime, mochisalactam, thienamycin, sulfazecin, meropenam, imepenam, ertapenam and aztreonam.

Examples of the antipyretic agents, analgesics and anti-inflammatory agents include, but are not limited to, salicylic acid, sulpyrine, flufenamic acid, diclofenac, indomethacin, morphine, pethidine hydrochloride, levorphanol tartrate and oxymorphone.

Examples of the antitussive expectorants include, but are not limited to, ephedrine hydrochloride, methylephedrine hydrochloride, noscapine hydrochloride, codeine phosphate, dihydrocodeine phosphate, allocramide hydrochloride, clofedanol hydrochloride, picoperidamine hydrochloride, chloperastine, protokylol hydrochloride, isoproterenol hydrochloride, sulbutamol sulfate and terbutaline sulfate.

Examples of the sedatives include, but are not limited to, chlorpromazine, prochlorperazine, trifltioperazine, atropine sulfate and methylscopolamine bromide.

Examples of the muscle relaxants include, but are not limited to, pridinol is methanesulfonate, tubocurarine chloride and pancuronium bromide.

Examples of the antiepileptics include, but are not limited to, phenytoin, ethosuximide, acetazolamide sodium and chlordiazepoxide.

Examples of the antiulcer agents include, but are not limited to, metoclopramide and histidine hydrochloride.

Examples of the antidepressants include, but are not limited to, imipramine, clomipramine, noxiptiline and phenerdine sulfate, amitriptyline HCl, amoxapine, butriptyline HCl, clomipramine HCl, desipramine HCl, dothiepin HCl, doxepin HCl, fluoxetine, gepirone, imipramine, lithium carbonate, mianserin HCl, milnacipran, nortriptyline HCl and paroxetine HCl; anti-muscarinic agents such as atropine sulphate and hyoscine; sedating agents such as alprazolam, buspirone HCl, chlordiazepoxide HCl, chlorpromazine, clozapine, diazepam, flupenthixol HCl, fluphenazine, flurazepam, lorazepam, mazapertine, olanzapine, oxazepam, pimozide, pipamperone, piracetam, promazine, risperidone, paliperidone, paliperidone palmitate, selfotel, seroquel, sulpiride, temazepam, thiothixene, triazolam, trifluperidol and ziprasidone; anti-migraine drugs such as alniditan and sumatriptan; beta-adrenoreptor blocking agents such as atenolol, carvedilol, metoprolol, nebivolol and propranolol; anti-Parkinsonian drugs such as bromocryptine mesylate, levodopa and selegiline HCl; opioid analgesics such as buprenorphine HCl, codeine, dextromoramide and dihydrocodeine; parasympathomimetics such as galanthamine, neostigmine, physostymine, tacrine, donepezil, ENA 713 (exelon) and xanomeline; and vasodilators such as amlodipine, buflomedil, amyl nitrite, diltiazem, dipyridamole, glyceryl trinitrate, isosorbide dinitrate, lidoflazine, molsidomine, nicardipine, nifedipine, oxpentifylline and pentaerythritol tetranitrate.

Examples of the anti-allergic agents include, but are not limited to, diphenhydramine hydrochloride, chlorpheniramine maleate, tripelenamine hydrochloride, methdilazine hydrochloride, clemizole hydrochloride, diphenylpyraline hydrochloride and methoxyphenamine hydrochloride.

Examples of the cardiotonics include, but are not limited to, trans-paioxocamphor, theophyllol, aminophylline and etilefrine hydrochloride.

Examples of the antiarrhythmic agents include, but are not limited to, propranol, alprenolol, bufetolol and oxprenolol.

Examples of the vasodilators include, but are not limited to, oxyfedrine hydrochloride, diltiazem, tolazoline hydrochloride, hexobendine and bamethan sulfate.

Examples of the hypotensive diuretics include, but are not limited to, hexamethonium bromide, pentolinium, mecamylamine hydrochloride, ecarazine hydrochloride and clonidine.

Examples of the antidiabetics include, but are not limited to, glymidine sodium, glipizide, fenformin hydrochloride, buformin hydrochloride and metformin.

Examples of the antihyperlipidemic agents include, but are not limited to, pravastatin sodium, simvastatin, clinofibrate, clofibrate, simfibrate and bezafibrate.

Example of the anticoagulant includes, but is not limited to, heparin sodium.

Examples of the hemolytics include, but are not limited to, thromboplastin, thrombin, menadione sodium hydrogen sulfite, acetomenaphthone, .epsilon-aminocaproic acid, tranexamic acid, carbazochrome sodium sulfonate and adrenochrome monoaminoguanidine methanesulfonate.

Examples of the antituberculosis agents include, but are not limited to, isoniazid, ethambutol and p-aminosalicylic acid.

Examples of the hormones include, but are not limited to, predonizolone, predonizolone sodium phosphate, dexamethasone sodium sulfate, betamethasone sodium phosphate, hexestrol phosphate, hexestrol acetate and methimazole.

Examples of the narcotic antagonists include, but are not limited to, levallorphan tartrate, nalorphine hydrochloride and naloxone hydrochloride.

Example of the bone resorption suppressor includes, but is not limited to, ipriflavone.

Examples of the osteogenesis promoters include, but are not limited to, polypeptides such as BMP, PTH, TGF-beta. and IGF-1, and (2R,4S)-(−)-N-[4-(diethoxyphosphorylmethyl)phenyl]-1,2,4,5-tetrahydro-4-methyl-7,8-methylenedioxy-5-oxo-3-benzothiepine-2-carboxamide and 2-(3-pyridyl)-ethane-1,1-diphosphonic acid.

Examples of the angiogenesis suppressors include, but are not limited to, angiogenesis-suppressing steroid, fumagillin and fumagillol derivatives.

The active ingredient(s) may be used as such or as a pharmacologically acceptable salt (e.g., salts formed with inorganic acids such as hydrochloric acid, sulfuric acid and nitric acid, and salts formed with organic acids such as carbonic acid and succinic acid, when the physiologically active substance has a basic group such as the amino group; salts formed with inorganic bases exemplified by alkali metals such as sodium and potassium, salts formed with organic base compounds exemplified by organic amines such as triethylamine, and basic amino acids such as is arginine, when the physiologically active substance has an acidic group such as the carboxy group).

According to embodiments of the present disclosure, the medium of the step (a) is selected from the group consisting of, silicon oil, sorbitan monooleate, polysorbate 20, polysorbate 40, polysorbate 60, polysorbate 65, polysorbate 80, vegetable oil, paraffine oil, polyvinyl alcohol (PVA), polyvinyl pyrrolidone (PVA), carboxyvinyl polymer (CVP), polyvinyl methyl ether (PVME), hydroxyethyl celluloses and poly(sodium acrylate) (PA), fatty acids, sodium lauryl sulfate and alpha olefin sulfonate. In one embodiment, the medium of the step (a) is PVA.

In one particular embodiment, to prepare the first mixture of the step (b), the active ingredient, which is in the state of an aqueous solution, is mixed with a non-aqueous solution of a polymer and a solvent at a temperature of 4 to 40° C. and a speed between at least 7,000 rpm. The first mixture is then allowed to come into contact with the continuous phase of the medium of the step (a), e.g., the continuous phase of PVA, in the conduit, to form a second mixture.

According to embodiments of the present disclosure, the polymer of the step (b) may be selected from the group consisting of, polyesters, polylactides, polyglycolides, poly(d,l-lactide-co-glycolide), polycaprolactones, polydioxannones, polycarbonates, polyhydroxybutyrates, polyalkyene oxalates, polyanhydrides, polyamides, polyesteramides, polyurethanes, polyacetals, polyketals, polyorthocarbonates, polyphosphazenes, polyhydroxyvalerates, polyalkylene succinates, poly(malic acid), poly(amino acids), chitin, chitosan, gelatin, polyorthoesters, polyethylene-polypropylene glycol copolymers, block polymer of polylactides-glycolides with polyethyleneglycol, terpolymers, block copolymers, branched copolymers, polyorthoester, polyanhydride, polyhydroxybutyric acid, polycaprolactone, polyalkylcarbonate, a copolymer or a simple mixture of two or more polymer groups, a copolymer of one of the above-mentioned polymers and polyethylenglycol (PEG), a polymer-sugar complex where a sugar is coupled with one of the above-mentioned polymers or the copolymers, and mixtures thereof. According to certain embodiment of the present disclosure, the polymer is poly(d,l-lactide-co-glycolide) having a molar ratio of lactide to glycolide in the range of about 85:15 to about 50:50. In one embodiment of the present disclosure, the polymer is polylactides (PLA) and modified poly(d,l-lactide-co-glycolide) (PLGA) or polylactides (PLA) end-group by acid or ester.

According to certain embodiment of the present disclosure, the solvent of the step (b) is any of, dichloromethane (DCM), N-methyl-2-pyrrolidone (NMP), aliphatic hydrocarbons, methane (CH4), ethane (C2H6), propane (C3H8), butane (C4H10), pentane (C5H12), hexane (C6H14), heptane (C7H16), octane (C8H18), acetone, acetic acid, chloroform, ethyl acetate, ethyl formate, methyl ketone, ethyl ketone, methyl isobutyl ketone, petroleum ether, 2-pyrrolidone, propylene carbonate, ethylene carbonate, dimethyl carbonate, 2-ethyoxylyl acetate, methyl acetate, ethyl lactate, ethyl butyrate, diethyl malonate, diethyl glutonate, tributyl citrate, diethyl succinate, tributyrin, isopropyl myristate, dimethyl adipate, dimethyl succinate, dimethyl oxalate, dimethyl citrate, triethyl citrate, acetyl tributyl citrate, glyceryl triacetate, methyl ethyl ketone, solketal, glycerol formal, glycofurol, dimethylformamide, dimethylacetamide, dimethylsulfoxide (DMSO), dimethylsulfone, tetrahydrofuran, epsilon-caprolactone, butyrolactone, capro lactam, N,N-dimethyl-m-toluamide, I-dodecylazacycloheptan-2-one, benzyl alcohol, benzyl benzoate, triaetin or a mixture thereof.

In one specific embodiment, in the step (b), the aqueous solution of the active ingredient is mixed with a non-aqueous solution of polylactic acid in the DCM, to form the first mixture. The first mixture is allowed to come into contact with the continuous phase of the medium in the conduit, thereby forming the second mixture.

In one embodiment, in the step (c), the second mixture of the step (b) then proceeds to enter the mixer, and is subject to a shear rate of 0.010/s to 0.300/s and a temperature of 4 to 40° C. in the mixer, and is continued to circulate in the in-line recirculating mixing system at a constant flow rate of 1 mL/min to 3000 L/min until the desired microparticles are formed.

Many of the attendant features and advantages of the present disclosure will becomes better understood with reference to the following detailed description considered in connection with the accompanying drawings.

In accordance with common practice, the various described features/elements are not drawn to scale but instead are drawn to best illustrate specific features/elements relevant to the present invention. Also, like reference numerals and designations in the various drawings are used to indicate like to elements/parts.

### BRIEF DESCRIPTION OF THE DRAWINGS

The present description will be better understood from the following detailed description read in light of the accompanying drawings, where:

FIG. 1 shows a preparation of microparticles disclosed in U.S. Pat. No. 6,534,094;

FIG. 2 is a schematic drawing illustrating an in-line recirculating mixing system 100 for use in the preparation method according to one embodiment of this invention;

FIG. 3 shows scanning electron microscopy (SEM) photographs of microparticles from batch 5, (A) surface, 100 k; (B) surface, 500 k; (C) surface, 2,000 k, and (D) cross-section, 2,000 k in accordance with one embodiment of the present disclosure; and

FIG. 4 is a line graph illustrating the result of the in vitro release profiles of Batch 5.

Like reference numerals are used to designate like parts in the accompanying drawings.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard is deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attaching claims are approximations that can vary as desired. At the very least, each numerical parameter should at least be construed in light of the number of reported significant digits and by applying ordinary rounding techniques.

As used herein, the term “active pharmaceutical ingredient(s)” refers to, physiologically active peptides, antitumor agents, antibiotics, antipyretic agents, analgesics, anti-inflammatory agents, antitussive expectorants, sedatives, muscle relaxants, antiepileptics, antiulcer agents, antidepressants, anti-allergic agents, cardiotonics, antiarrhythmic agents, vasodilators, hypotensive diuretics, antidiabetics, antihyperlipidemic agents, anticoagulants, hemolytics, antituberculosis agents, hormones, narcotic antagonists, bone resorption suppressors, osteogenesis promoters or angiogenesis inhibitors.

Examples of the physiologically active peptides may be selected from the group consisting of, growth hormone releasing peptide (GHRP), luteinizing hormone-releasing hormone (LHRH), bombesin, gastrin releasing peptide (GRP), calcitonin, bradykinin, galanin, melanocyte stimulating hormone (MSH), growth hormone releasing factor (GRF), amylin, tachykinins, secretin, parathyroid hormone (PTH), enkephalin, endothelin, calcitonin gene releasing peptide (CGRP), neuromedins, parathyroid hormone related protein (PTHrP), glucagon, neurotensin, peptide YY (PYY), glucagon-like peptide-1 (GLP1), liraglutide, exenatide, lixisenatide, albiglutide, dulaglutide, taspoglutide, semaglutide, vasoactive intestinal peptide (VIP), pituitary adenylate cyclase activating peptide (PACAP), motilin, substance P, neuropeptide Y (NPY), thyroid stimulating hormone (TSH), insulin, somatostatin, somatostatin derivative, growth hormones, prolactin, adrenocorticotropic hormone (ACTH), ACTH derivatives (e.g., ebiratide), thyrotropin-releasing hormone and salts and derivatives thereof, thyroid-stimulating luteinizing hormone (LH), follicle-stimulating hormone (FSH), vasopressin, vasopressin derivative, oxytocin, calcitonin, gastrin, pancreozymin, cholecystokinin, angiotensin, human placental lactogen, human chorionic gonadotropin (HCG), enkephalin, enkephalin derivatives, endorphin, kyotorphin, interferons, interleukins, tuftsin, thymopoietin, thymosin, thymostimulin, thymic humoral factor (THF), blood thymic factor (FTS) and derivative thereof, other thymic factors, tumor necrosis factor (TNF), colony-stimulating factors (e.g., CSF, GCSF, GMCSF, MCSF), dynorphin, caerulein, bradykinin, urokinase, asparaginase, kallikrein, insulin-like growth factors (IGF-I, IGF-II), nerve growth factor (NGF), cell growth factors (e.g., EGF, TGF-.alpha., TGF-.beta., PDGF, acidic FGF, basic FGF), bone morphogenic factor (BMP), nerve nutrition factors (e.g., NT-3, NT-4, CNTF, GDNF, BDNF), blood coagulation factors VIII and IX, lysozyme chloride, polymixin B, colistin, gramicidin, bacitracin, erythropoietin (EPO), thrombopoietin (TPO), and endothelin-antagonistic peptides and analogs and fragments thereof. In one preferred embodiment, the active ingredient(s) is LH-RH or an analog thereof, still more preferably leuprorelin or leuprorelin acetate. In one preferred embodiment, the active ingredient(s) is GLP-1 or an analog thereof, still more preferably exenatide.

Examples of the antitumor agents include, but are not limited to, bleomycin, methotrexate, actinomycin D, mitomycin C, binblastin sulfate, bincrystin sulfate, daunorubicin, adriamycin, neocartinostatin, cytosinearabinoside, fluorouracil, tetrahydrofuryl-5-fluorouracil, krestin, Picibanil, lentinan, levamisole, Bestatin, azimexon, glycyrrhizin, polyl:C, polyA:U and polyICLC.

Examples of the antibiotics include, but are not limited to, gentamicin, dibekacin, Kanendomycin, lividomycin, tobramycin, amikacin, fradiomycin, sisomycin, tetracycline hydrochloride, oxytetracycline hydrochloride, rolitetracycline, doxycycline hydrochloride, ampicillin, piperacillin, ticarcillin, cefalothin, cefaloridine, cefotiam, cefsulodin, cefmenoxime, cefmetazole, cefazolin, cefotaxime, cefoperazon, ceftizoxime, mochisalactam, thienamycin, sulfazecin and aztreonam.

Examples of the anti-pyretic agents, analgesics and anti-inflammatory agents include, but are not limited to, salicylic acid, sulpyrine, flufenamic acid, diclofenac, indomethacin, morphine, pethidine hydrochloride, levorphanol tartrate and oxymorphone.

Examples of the anti-tussive expectorants include, but are not limited to, is ephedrine hydrochloride, methylephedrine hydrochloride, noscapine hydrochloride, codeine phosphate, dihydrocodeine phosphate, allocramide hydrochloride, clofedanol hydrochloride, picoperidamine hydrochloride, chloperastine, protokylol hydrochloride, isoproterenol hydrochloride, sulbutamol sulfate and terbutaline sulfate.

Examples of the sedatives include, but are not limited to, chlorpromazine, prochlorperazine, trifltioperazine, atropine sulfate and methylscopolamine bromide.

Examples of the muscle relaxants include, but are not limited to, pridinol methanesulfonate, tubocurarine chloride and pancuronium bromide.

Examples of the anti-epileptics include, but are not limited to, phenytoin, ethosuximide, acetazolamide sodium and chlordiazepoxide.

Examples of the antiulcer agents include, but are not limited to, metoclopramide and histidine hydrochloride.

Examples of the anti-depressants include, but are not limited to, imipramine, clomipramine, noxiptiline and phenerdine sulfate, amitriptyline HCl, amoxapine, butriptyline HCl, clomipramine HCl, desipramine HCl, dothiepin HCl, doxepin HCl, fluoxetine, gepirone, imipramine, lithium carbonate, mianserin HCl, milnacipran, nortriptyline HCl and paroxetine HCl; anti-muscarinic agents such as atropine sulphate and hyoscine; sedating agents such as alprazolam, buspirone HCl, chlordiazepoxide HCl, chlorpromazine, clozapine, diazepam, flupenthixol HCl, fluphenazine, flurazepam, lorazepam, mazapertine, olanzapine, oxazepam, pimozide, pipamperone, piracetam, promazine, risperidone, paliperidone, paliperidone palmitate, selfotel, seroquel, sulpiride, temazepam, thiothixene, triazolam, trifluperidol and ziprasidone; anti-migraine drugs such as alniditan and sumatriptan; beta-adrenoreptor blocking agents such as atenolol, carvedilol, metoprolol, nebivolol and propranolol; anti-Parkinsonian drugs such as bromocryptine mesylate, levodopa and selegiline HCl; opioid analgesics such as buprenorphine HCl, codeine, dextromoramide and dihydrocodeine; parasympathomimetics such as galanthamine, neostigmine, physostymine, tacrine, donepezil, ENA 713 (exelon) and xanomeline; and vasodilators such as amlodipine, buflomedil, amyl nitrite, diltiazem, dipyridamole, glyceryl trinitrate, isosorbide dinitrate, lidoflazine, molsidomine, nicardipine, nifedipine, oxpentifylline and pentaerythritol tetranitrate.

Examples of the anti-allergic agents include, but are not limited to, diphenhydramine hydrochloride, chlorpheniramine maleate, tripelenamine hydrochloride, methdilazine hydrochloride, clemizole hydrochloride, diphenylpyraline hydrochloride and methoxyphenamine hydrochloride.

Examples of the cardiotonics include, but are not limited to, trans-paioxocamphor, theophyllol, aminophylline and etilefrine hydrochloride.

Examples of the antiarrhythmic agents include propranol, alprenolol, bufetolol and oxprenolol.

Examples of the vasodilators include, but are not limited to, oxyfedrine hydrochloride, diltiazem, tolazoline hydrochloride, hexobendine and bamethan sulfate.

Examples of the hypotensive diuretics include, but are not limited to, hexamethonium bromide, pentolinium, mecamylamine hydrochloride, ecarazine hydrochloride and clonidine.

Examples of the antidiabetics include, but are not limited to, glymidine sodium, glipizide, fenformin hydrochloride, buformin hydrochloride and metformin.

Examples of the antihyperlipidemic agents include, but are not limited to, pravastatin sodium, simvastatin, clinofibrate, clofibrate, simfibrate and bezafibrate.

Example of the anticoagulant includes, but is not limited to, heparin sodium.

Examples of the hemolytics include, but are not limited to, thromboplastin, thrombin, menadione sodium hydrogen sulfite, acetomenaphthone, .epsilon.-aminocaproic acid, tranexamic acid, carbazochrome sodium sulfonate and adrenochrome monoaminoguanidine methanesulfonate.

Examples of the antituberculosis agents include, but are not limited to, isoniazid, ethambutol and p-aminosalicylic acid.

Examples of the hormones include, but are not limited to, predonizolone, predonizolone sodium phosphate, dexamethasone sodium sulfate, betamethasone sodium phosphate, hexestrol phosphate, hexestrol acetate and methimazole.

Examples of the narcotic antagonists include, but are not limited to, levallorphan tartrate, nalorphine hydrochloride and naloxone hydrochloride.

Example of the bone resorption suppressor includes, but is not limited to, ipriflavone.

Examples of the osteogenesis promoters include, but are not limited to, polypeptides such as BMP, PTH, TGF-.beta. and IGF-1, and (2R,4S)-(−)-N-[4-(diethoxyphosphorylmethyl)phenyl]-1,2,4,5-tetrahydro-4-methyl-7,8-methylenedioxy-5-oxo-3-benzothiepine-2-carboxamide and 2-(3-pyridyl)-ethane-1,1-diphosphonic acid.

Examples of the angiogenesis suppressors include, but are not limited to, angiogenesis-suppressing steroid, fumagillin and fumagillol derivatives.

The active ingredient(s) may be used as such or as a pharmacologically acceptable salt e.g., salts formed with inorganic acids such as hydrochloric acid, sulfuric acid and nitric acid, and salts formed with organic acids such as carbonic acid and succinic acid, when the physiologically active substance has a basic group such as the amino group; salts formed with inorganic bases exemplified by alkali metals such as sodium and potassium, salts formed with organic base compounds exemplified by organic amines such as triethylamine, and basic amino acids such as arginine, when the physiologically active substance has an acidic group such as the carboxy group.

As used herein, an “excipient” is one that is suitable for use with the subjects without undue adverse side effects (such as toxicity, irritation, and allergic response) commensurate with a reasonable benefit/risk ratio. Also, each excipient must be “acceptable” in the sense of being compatible with the other ingredients of the microparticles.

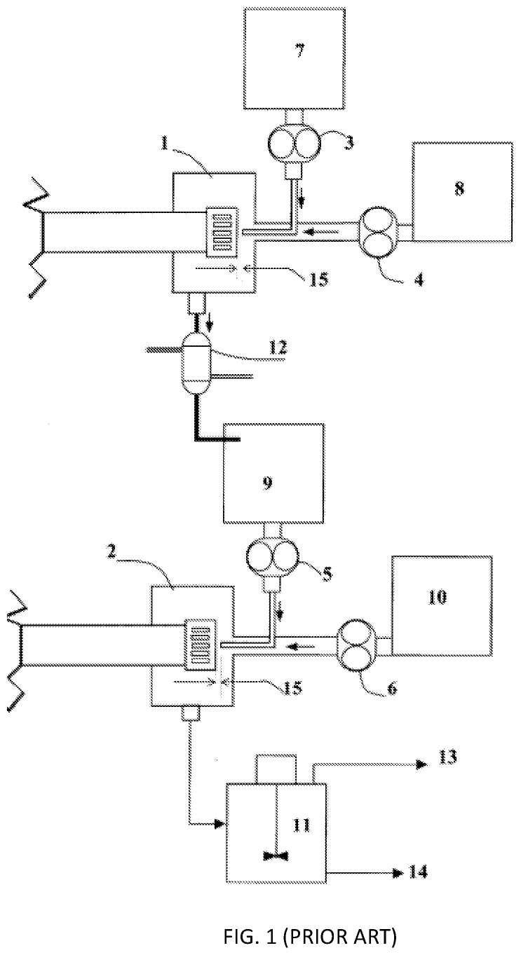

In the method disclosed in U.S. Pat. No. 6,534,094, the point of injection, or the point where the active ingredient(s) encounters the polymer solution, is inside of the homogenizer; and the size of the thus produced particle is subsequently adjusted by sieving. As evident from FIG. 1, the container where the active peptide aqueous solution 7 resides has a conduit that extends into the first mixer 1, allowing the respective solutions in the container 7 and 8 to be mixed in the first mixer. Similarly, the container for cooling the first emulsion 9 also has a conduit that extends into the second mixer 2, thereby allowing the respective solutions in the container 9 and 10 to be mixed in the second mixer 2. Furthermore, the system disclosed in U.S. Pat. No. 6,534,094 is a batch-type system, and not a continuous system. Further according to FIG. 1, the solution processed in the second mixer 2 is transferred to the vacuum evaporator 11, instead of being continuously circulated in the system.

The present invention, however, is directed to a novel method for producing microparticles of an active ingredient(s), in which the point of injection of an active ingredient(s)/polymer solution does not reside in the homogenizer (i.e. mixer), but in the conduit. The difference in the location where the active ingredient(s) comes into contact with the polymeric component has not only resulted in a more uniform distribution in the size of the final product (i.e., polymeric microparticles), but also eliminating the need of subsequent sieving step that leads to an increase in the production yield of the present method. Thus, the method disclosed herein is not a batch-type preparation method as that of U.S. Pat. No. 6,534,094, but a continuous is preparation of polymeric microparticles that is carried out in an in-line recirculating mixing system.

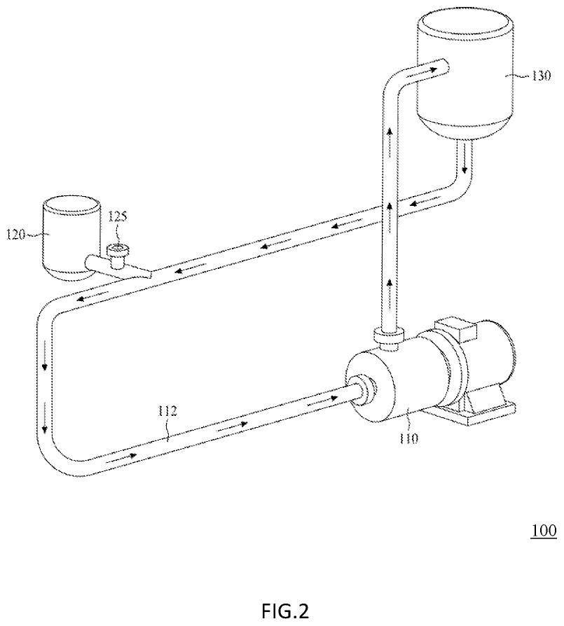

FIG. 2 is a schematic drawing illustrating an in-line recirculating mixing system 100 used in the present method. The in-line recirculating mixing system 100 comprises in its structure, a mixer 110 and a conduit 112 coupling to the inlet and the outlet of the mixer 110, thereby forming a closed system. Optionally, the in-line recirculating mixing system 100 may further comprise a tank 120 and a reservoir 130, respectively coupled with the conduit 112, wherein the tank 120 acts as a storage for the solution to be injected to the fluid in the in-line recirculating mixing system 100, whereas the reservoir 130 acts as a storage for the fluid in the in-line recirculating mixing system 100.

Before starting the production process, a medium is independently prepared and introduced into the in-line recirculating mixing system 100 to form a continuous phase, so as to remove any residual air in the in-line recirculating mixing system 100. The medium may be introduced into the system 100 from any suitable location. For example, the medium may enter the in-line recirculating mixing system 100 from a site (denoted as “P” in FIG. 2) located at the conduit 112. Alternatively, the medium may enter the in-line recirculating mixing system 100 directly from the mixer 110. Suitable examples of the medium to be used in the present method include, but are not limited to, silicon oil, sorbitan monooleate, polysorbate 20, polysorbate 40, polysorbate 60, polysorbate 65, polysorbate 80, vegetable oil, paraffine oil, polyvinyl alcohol (PVA), polyvinyl pyrrolidone, carboxyvinyl polymer (CVP), polyvinyl methyl ether (PVME), hydroxyethyl celluloses and poly(sodium acrylate) (PA), fatty acids, sodium lauryl sulfate or alpha olefin sulfonate. In one preferred embodiment, PVA is introduced into the system 100 from P, and circulates therein to form a continuous phase.

Next, a first mixture of an active ingredient, a polymer and a solvent is prepared. Typically, the first mixture is formed by mixing an aqueous solution of the active ingredient with a non-aqueous solution of the polymer. In certain embodiment, the active ingredient is suspended or dissolved in an aqueous solvent, e.g., water to to form the aqueous solution of the active ingredient, while the polymer is suspended or dissolved in an organic solvent, e.g., dichloromethane (DCM), to form a non-aqueous solution of the polymer. The aqueous solution of the active ingredient is then mixed with the non-aqueous solution of the polymer to produce the first mixture.

In one embodiment, an aqueous solution of LHRH (about 35-50% by weight) is prepared, in which the aqueous solution is prepared by dissolving LHRH in water. In another embodiment, an aqueous solution of GLP-1 (about 5-15% by weight) is prepared, in which the aqueous solution is prepared by dissolving GLP-1 in water.

The non-aqueous solution is prepared by dissolved biodegradable polymer in an organic solvent at a weight ratio of 1:1 to 1:20, such as 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 and 1:20; and stirred until a clear solution is formed. Suitable examples of the biodegradable polymer useful in the present method include, but are not limited to, poly(d,l-lactide-co-glycolide) (PLGA) having a molar ratio of lactide to glycolide in the range of about 85:15 to about 50:50 or polylactides (PLA). Suitable examples of organic solvent useful in the present method include, but are not limited to, dichloromethane (DCM), ethyl acetate, acetonitrile, heptane, hexane, petroleum ether. According to preferred embodiments relating to the production of LHRH microparticles, the PLA is mixed with DCM in a weight ratio of about 1:1.7. According to other preferred embodiments relating to the production of in GLP-1 microparticles, the PLGA is mixed with DCM in a weight ratio of about 1:15.7.

According to embodiments of the present disclosure, the first mixture may be prepared in a mixer or a homogenizer that is independent from the mixer or homogenizer in the in-line recirculating mixing system 100. Alternatively, in some embodiments, the mixer or the homogenizer used to prepare the first mixture is incorporated into the in-line recirculating mixing system 100 of this invention. In one embodiment, the first mixture is a water/oil emulsion, wherein the aqueous solution and non-aqueous solution are mixed in a volume ratio of about 1:1 to 1:100 at a speed of about at least 6,000 rpm; preferably, in a volume ratio of about 1:2 to 1:50 at a speed of about 6,200-20,000 rpm; and more preferably, in a volume ratio of about 1:4 to 1:25 at a speed of about 6,500-12,000 rpm. For example, the aqueous solution and the non-aqueous solution are mixed in the ratio of about 1:5 to 1:12 at a speed of at least 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, 10,100, 10,200, 10,300, to 10,400, 10,500, 10,600, 10,700, 10,800, 10,900, 11,000, 11,100, 11,200, 11,300, 11,400, 11,500, 11,600, 11,700, 11,800, 11,900, 12,000, 12,100, 12,200, 12,300, 12,400, 12,500, 12,600, 12,700, 12,800, 12,900, 13,000 rpm or above. Preferably, the aqueous solution and the non-aqueous solution are mixed at the temperature of about 0-40° C., such as 0, 1, 2, 3, 4, 5, 6, 7, 8, 9, 10, 11, 12, 13, 14, 15, 16, 17, 18, 19, is 20, 21, 22, 23, 24, 25, 26, 27, 28, 29, 30, 31, 32, 33, 34, 35, 36, 37, 38, 39 or 40° C.

After establishing the continuous phase of the medium, the first mixture described above is stored within the tank 120, and is subsequently introduced into the in-line recirculating mixing system 100 using a pump 125 from a site (denotedas “P” in FIG. 2) located at the conduit 112, such that it comes into contact with the already circulating medium therein, thereby forming a second mixture in the conduit 112. In one embodiment, the pump 125 in the in-line recirculating mixing system 100 is configured to control the flow rate of the first mixture entering the conduit 112. It should be noted that, at this point, the first mixture has not yet reached the mixer 110, and has first come into contact with the medium, which is already circulating in the in-line recirculating mixing system 100 as a continuous phase, thereby forms a second mixture in the conduit 112, before entering into the mixer 110. In another embodiment, a syringe is employed to replace the tank 120 in the in-line recirculating mixing system 100.

According to preferred embodiments, the first mixture and the continuous phase of the medium come into contact in a volume ratio of about 1:20 to 1:1,200 at a speed at least 7,000 rpm; preferably, in a volume ratio of about 1:30 to 1:1,000 at a speed of at least 7,000 rpm; and more preferably, in a volume ratio of about 1:40 to 1:800 at a speed of at least 7,000 rpm. For example, the first mixture and the medium come into contact in the ratio of about 1:20, 1:30, 1:40, 1:50, 1:60, 1:70, 1:80, 1:90, 1:100, 1:110, 1:120, 1:130, 1:140, 1:150, 1:160, 1:170, 1:180, 1:190, 1:200, 1:210, 1:220, 1:230, 1:240, 1:250, 1:260, 1:270, 1:280, 1:290, 1:300, 1:310, 1:320, 1:330, 1:340, 1:350, 1:360, 1:370, 1:380, 1:390, 1:400, 1:410, 1:420, 1:430, 1:440, 1:450, 1:460, 1:470, 1:480, 1:490, 1:500, 1:510, 1:520, 1:530, 1:540, 1:550, 1:560, 1:570, 1:580, 1:590, 1:600, 1:610, 1:620, 1:630, 1:640, 1:650, 1:660, 1:670, 1:680, 1:690, 1:700, 1:710, 1:720, 1:730, 1:740, 1:750, 1:760, 1:770, 1:780, 1:790, 1:800, 1:810, 1:820, 1:830, 1:840, 1:850, 1:860, 1:870, 1:880, 1:890, 1:900, 1:910, 1:920, 1:930, 1:940, 1:950, 1:960, 1:970, 1:980, 1:990, 1:1,000, 1:1,010, 1:1,020, 1:1,030, 1:1,040, 1:1,050, 1:1,060, 1:1,070, 1:1,080, 1:1,090, 1:1,100, 1:1,110, 1:1,120, 1:1,130, 1:1,140, 1:1,150, 1:1,160, 1:1,170, 1:1,180, 1:1,190 or 1:1,200; at a speed of at least 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, 10,100, 10,200, 10,300, 10,400, 10,500, 10,600, 10,700, 10,800, 10,900, 11,000, 11,100, 11,200, 11,300, 11,400, 11,500, 11,600, 11,700, 11800, 11,900, 12,000, 12,100, 12,200, 12,300, 12,400, 12,500, 12,600, 12,700, 12,800, 12,900, 13,000, 13,100, 13,200, 13,300, 13,400, 13,500, 13,600, 13,700, 13,800, 13,900, 14,000, 14,100, 14,200, 14,300, 14,400, 14,500, 14,600, 14,700, 14,800, 14,900, 15,000, 15,100, 15,200, 15,300, 15,400, 15,500, 15,600, 15,700, 15,800, 15,900, 15,000, 16,100, 16,200, 16,300, 16,400, 16,500, 16,600, 16,700, 16,800, 16,900, 17,000, 17,100, 17,200, 17,300, 17,400, 17,500, 17,600, 17,700, 17,800, 17,900, 18,000, 18,100, 18,200, 18,300, 18,400, 18,500, 18,600, 18,700, 18,800, 18,900, 19,000, 19,100, 19,200, 19,300, 19,400, 19,500, 19,600, 19,700, 19,800, 19,900, 20,000, 20,100, 20,200, 20,300, 20,400, 20,500, 20,600, 20,700, 20,800, 20,900, 21,000, 21,100, 21,200, 21,300, 21,400, 21,500, 21,600, 21,700, 21,800, 21,900, 22,000, 22,100, 22,200, 22,300, 22,400, 22,500, 22,600, 22,700, 22,800, 22,900, 23,000 rpm or above.

Once the second mixture is formed in the conduit 112, it then proceeds to enter the mixer 110, and circulate in the in-line recirculating mixing system 100 at a constant flow rate of 1 mL-3,000 L/min, such as 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 160, 210, 260, 310, 360, 410, 460, 510, 560, 610, 660, 710, 760, 810, 860, 910, 960 or 1,000 mL/min, and 1, 2, 3, 4, 5, 6, 7, 8, 9, 10, 11, 12, 13, 14, 15, 16, 17, 18, 19, 20 20, 25, 30, 35, 40, 45, 50, 55, 60, 65, 70, 75, 80, 85, 90, 95, 100, 110, 160, 210, 260, 310, 360, 410, 460, 510, 560, 610, 660, 710, 760, 810, 860, 910, 960, 1,000, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000 L/min, until the desired microparticles of the active ingredient(s) are formed. It is worthy to note that the immediately formed second mixture (i.e., the second mixture that forms in the conduit 112) is composed by particles having relatively lose structures that tend to aggregate. Once the second mixture enters the mixer 110, it is subject to shear rate of 0.010/s to 0.300/s and a temperature of 4 to 40° C. in the mixer, such as 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40° C. According to one embodiment, the second mixture is subject to a shear rate of 0.010/s-0.300/s in the mixer, such as 0.010, 0.011, 0.012, 0.013, 0.014, 0.015, 0.016, 0.017, 0.018, 0.019, 0.020, 0.021, 0022, 0.023, 0.024, 0.025, 0.026, 0.027, 0.028, 0.029, 0.030, 0.035, 0.040, 0.045, 0.050, 0.055, 0.060, 0.065, 0.070, 0.075, 0.080, 0.085, 0.090, 0.095, 0.100, 0.105, 0.110, 0.115, 0.120, 0.125, 0.130, 0.135, 0.140, 0.145, 0.150, 0.155, 0.160, 0.165, 0.170, 0.175, 0.180, 0.185, 0.190, 0.195, 0.200, is 0.205, 0.210, 0.215, 0.220, 0.225, 0.230, 0.235, 0.240, 0.245, 0.250, 0.255, 0.260, 0.265, 0.270, 0.275, 0.280, 0.285, 0.290, 0.295, 0.300. With the actions of both the shear rate in the mixer and the continuous phase of the medium, the sizes of the particles in the second mixture are further reduced, while their structures gradually condense due to the continued removal of the solvent from the particles by the extraction action conferred by the medium. The particles are allowed to continuously circulate in the system 100, until they exhibit the desired property, i.e., uniform distribution is particle size, and rigid enough to exist in microparticles without forming an aggregate. It should be noted that the step of circulating the second mixture in the system 100 shall be allowed to proceed with a desired period of times, such as at least 10 seconds, so that microparticles having a relatively uniform distribution in both sizes and structures are produced. Accordingly, the thus-produced microparticles have a narrow particle size distribution. The experimental data provided below demonstrate that this step of “reducing the particle size of soft microparticles” is advantageous so that said narrow particle size distribution could be achieved without sieving. Additionally, the present invention is also advantage in high yield of the microparticles.

In one example, the yield of the present invention is about 60%. According to various embodiments of present invention, the average diameter of the microparticles produced by the process of this invention is less than 200 μm. In one embodiment, the average diameter of the microparticles is about 1 μm to 20 μm. For example, the average diameter of the microparticles is about 1, 2, 3, 4, 5, 6, 7, 8, 9, 10, 11, 12, 13, 14, 15, 16, 17, 18, 19 or 20 μm.

Moreover, the microparticles produced by the method of the present invention are quite stable. Stability test demonstrates that the microparticles are stable for at least three months. Furthermore, the producing method of this invention may result in a long-term release depot; such as one week to one year.

The microparticles prepared by the present invention are fine particles, in which each particle comprises at least one active ingredient (e.g., drugs) and at least one polymer. In some cases, each microparticles has a core constituted by one active ingredient; in other cases, each microparticles has a core constituted by more than one active ingredients, such as 2, 3 or 4 active ingredients. According to some embodiments of the present invention, the microparticles include microcapsules containing one core in each particle. In another embodiment, microparticles include microcapsules containing multiple cores in each particle.

The following examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

### Example 1

1.1 Formulations for Forming Microparticles of Active Pharmaceutical Substance

In these examples, microparticles were prepared from formulations as provided in Table 1, and their respective dissolution duration (for example, one week or three month depot) were subsequently investigated. Specifically, formulations A, B and C were respectively one-month depot, three-month depot and six-month depot of leuprolide acetate, while formulation D was one-week depot of exenatide, which is a human GLP-1 analog for treating diabetes. Further, formulation E was two-week depot of risperidone for treating schizophrenia.

TABLE 1
Microparticle Formulations

| | Formulation A | | Formulation B | | Formulation C | |
|---|---|---|---|---|---|---|
| Polymer | PLGA 75/25 | 75% | PLA | 76.4% | PLA | 64.2% |
| | 8,400-14,000 | | 11,000-18,200 | | 19,000-27,000 | |
| | Carboxyl acid | | Carboxyl acid | | Carboxyl | |
| | end group | | end group | | acid end | |
| | | | | | group | |
| API | Leuprolide | 8.5% | Leuprolide | 8.6% | Leuprolide | 17% |
| | acetate | | acetate | | acetate | |
| Excipient | Mannitol | 15% | Mannitol | 15% | Mannitol | 15% |
| | Gelatin | 1.5% | | | Stearic acid | 3.8% |

| | |
|---|---|
| Formulation D | Formulation E |

| | | | | |
|---|---|---|---|---|
| Polymer | PLGA 50/50 | 93% | PLGA 75/25 | 61.9% |
| | 40,000-70,000 | | 110,000-150,000 | |
| | Carboxyl acid | | ester end group | |
| | end group | | | |
| API | Exenatide | 5% | Risperidone | 38.1% |
| Excipient | Sucrose | 2% | | |

API: Active Pharmaceutical Ingredient

1.2 Microparticles Prepared by the Continuous Circulating Process

1.2.1 1-Month Depot of Leuprolide Acetate

To prepare batches 1 to 3, the leuprolide acetate microparticles of batches 1 to 3 were prepared in the in-line recirculating mixing system as depicted in FIG. 2 using the parameters provided in Table 2. A typical protocol for preparing batch 1 is described as follows. Briefly, in this example, the 1-month leuprolide acetate of batch 1 is prepared using the formulation A disclosed in Table 1. 0.3 grams of leuprolide acetate and gelatin 0.05 g were dissolved in 0.3 grams of water for injection with magnetic agitation at 40-50° C. to produce an API solution. 2.0 grams of PLA were dissolved in 3.4 grams of dichloromethane (DCM) in the ultrasonic bath, followed by stirring to produce a polymer solution. The API solution was then added into the polymer solution, and formed a first mixture using the homogenizer at the speed of about 7,000 rpm for 5 minutes at 15° C. Then, 500 mL of 0.25% PVA solution was filtrated via the 0.2 μm filter and cooled to 18.5° C. The mixer 110 of the mixing system 100 was speeded up to a rotor speed of 15,000 rpm and 0.25% PVA solution was conducted into the in-line recirculating mixing system 100 to remove the air from the conduit 112. The first mixture was injected into the conduit 112 of the in-line recirculating mixing system 100. The first mixture and PVA solution were mixed in the conduit 112 before entering into the mixer 110 to form a second mixture. The second mixture was entered into the mixer 110 and circulated in the in-line recirculating mixing system 100 until the microparticles of the active ingredient(s) were formed, wherein the sizes of the microparticles were reduced in the mixer 110, and the microparticles were hardened by the solvent extraction in the conduit 112. The dichloromethane was further evaporated using a mechanical stirrer for 3 hours at room temperature. Next, the microparticles were collected by centrifugation at 2,000 g for 5 minutes, and washed for three times to remove the PVA solution. Mannitol solution was filtrated with 0.2 μm filter. Microparticles were resuspended by adding water, and mannitol solution was added into the microparticles suspension. The microparticles were stored at −80° C. until lyophilization.

TABLE 2
Condition for manufacturing microparticles of 1-month depot of
leuprolide acetate

| | | | | | Clearance | |
|---|---|---|---|---|---|---|
| | | | | | between | |
| | | Rotor | Rotor | | rotor and | Shear |
| | | speed | diameter | Tip speed | stator | rate |
| Batch | Formulation | (rpm) | (mm) | (m/s) | (mm) | (1/s) |
| 1 | A | 15,000 | 18 | 14.1 | 0.5 | 0.028 |
| 2 | A | 14,000 | 18 | 13.2 | 0.5 | 0.026 |
| 3 | A | 12,000 | 18 | 11.3 | 0.5 | 0.023 |

1.2.2 3-Month Depot of Leuprolide Acetate

To prepare batches 4 to 10, the leuprolide acetate microparticles of batches 4 to 10 were prepared in the in-line recirculating mixing system as depicted in FIG. 2 using the parameters provided in Table 3. A typical protocol for preparing batch 5 is described as follows. Briefly, in this example, the 3-month leuprolide acetate of batch 5 is prepared using the formulation B disclosed in Table 1. 0.3 grams of leuprolide acetate were dissolved in 0.3 grams of water for injection with magnetic agitation at 40-50° C. to produce an API solution. 2.0 grams of PLA were dissolved in 3.4 grams of dichloromethane (DCM) in the ultrasonic bath, followed by stirring to produce a polymer solution. The API solution was then added into the polymer solution, and formed first mixture using the homogenizer at the speed of about 7,000 rpm for 5 minutes at 15° C. Then, 500 mL of 0.5% PVA solution was filtrated via the 0.2 μm filter and cooled to 18.5° C. The mixer 110 of the mixing system 100 was to speeded up to a rotor speed of 22,500 rpm and 0.5% PVA solution was conducted into the in-line recirculating mixing system 100 to remove the air from the conduit 112. The first mixture was injected into the conduit 112 of the in-line recirculating mixing system 100. The first mixture and PVA solution were mixed in the conduit 112 before entering into the mixer 110 to form a second mixture. The second mixture is was entered into the mixer 110 and circulated in the in-line recirculating mixing system 100 until the microparticles of the active ingredient(s) were formed, wherein the sizes of microparticles were reduced in the mixer 110, and the microparticles were hardened by the solvent extraction in the conduit 112. The dichloromethane was further evaporated using a mechanical stirrer for 60 minutes at room temperature. Next, the microparticles were collected by centrifugation at 2,000 g for 5 minutes, and washed for three times to remove the PVA solution. Mannitol solution was filtrated with 0.2 μm filter. Microparticles were resuspended by adding water, and mannitol solution was added into the microparticles suspension. The microparticles were stored at −80° C. until lyophilization.

TABLE 3
Condition for manufacturing manufacturing microparticles of
3-month depot of leuprolide acetate

| | | | | | Clearance | |
|---|---|---|---|---|---|---|
| | | | | | between | |
| | | Rotor | Rotor | | rotor and | Shear |
| | | speed | diameter | Tip speed | stator | rate |
| Batch | Formulation | (rpm) | (mm) | (m/s) | (mm) | (1/s) |
| 4 | B | 15,000 | 18 | 13.3 | 0.5 | 0.028 |
| 5 | B | 22,500 | 18 | 20 | 0.5 | 0.042 |
| 6 | B | 9,000 | 38 | 17.9 | 0.15 | 0.119 |
| 7 | B | 10,230 | 38 | 20.3 | 0.15 | 0.136 |
| 8 | B | 20,000 | 18 | 18.8 | 0.5 | 0.038 |
| 9 | B | 17,000 | 18 | 16.0 | 0.5 | 0.032 |
| 10 | B | 16,000 | 18 | 15.1 | 0.5 | 0.030 |

1.2.3 6-Month Depot of Leuprolide Acetate

To prepare batches 11 to 13, the leuprolide acetate microparticles of batches 11 to 13 were prepared in the in-line recirculating mixing system as depicted in FIG. 2 using the parameters provided in Table 4. A typical protocol for preparing batch 11 is described as follows. Briefly, in this example, the 6-month leuprolide acetate of batch 11 was prepared by mixing all the ingredients of formulation C disclosed in Table 1. 0.225 grams of leuprolide acetate and 0.05 grams of stearic acid were dissolved in 1.3 grams of methanol with a continuous agitation using a magnetic stirrer at 40° C. to produce an API solution. 0.85 grams of PLA were dissolved in 2.6 grams of dichloromethane (DCM) in the ultrasonic bath with continuous stir to produce a polymer solution. The polymer solution was then added into the API solution with magnetic stirring at 30° C. to form a first mixture. Then, 250 mL of 0.5% PVA solution was filtrated via the 0.2 μm filter and cooled to 18° C. The mixer 110 of the mixing is system 100 was ramped up to a rotor speed of 7,000 rpm and 0.5% PVA solution was conducted into the in-line recirculating mixing system 100 to remove the air from the conduit 112. The first mixture was injected into the conduit 112 of the in-line recirculating mixing system 100. The first mixture and PVA solution were mixed in the conduit 112 before entering into the mixer 110 to form a second mixture. The second mixture then entered into the mixer 110 and circulated in the in-line recirculating mixing system 100 until microparticles of the active ingredient(s) were formed, and the sizes of microparticles were reduced in the mixer 110 and hardened due to the extraction of the solvent (i.e., DCM) from the circulated microparticles in the conduit 112. The solvent was further evaporated using a mechanical stirrer for 180 minutes at 18° C. Next, the microparticles were collected by centrifuging at 1,000×g for 10 minutes, then washed for three times to remove the residual PVA solution. Mannitol solution was filtered with 0.2 μm filter. Microparticles were re-suspended in water, and mannitol solution was added into the microparticles suspension. The microparticles were stored at −80° C. until lyophilization.

TABLE 4
Conditions for manufacturing microparticles of 6-month depot
of leuprolide acetate

| | | | | | Clearance | |
|---|---|---|---|---|---|---|
| | | Rotor | Rotor | Tip | between rotor | Shear |
| | | speed | diameter | speed | and stator | rate |
| Batch | Formulation | (rpm) | (mm) | (m/s) | (mm) | (1/s) |
| 11 | C | 7,000 | 18 | 6.6 | 0.5 | 0.013 |
| 12 | C | 12,000 | 18 | 11.3 | 0.5 | 0.023 |
| 13 | C | 17,000 | 18 | 16.0 | 0.5 | 0.032 |

1.2.4 Two-Week Depot of Risperidone

To prepare batches 14 to 16, the risperidone microparticles of batch 14 to 16 were prepared in the in-line recirculating mixing system as depicted in FIG. 2 using the parameters provided in Table 5. A typical protocol for preparing batch 15 is described as follows. Briefly, in this example, the risperidone microparticles of batch 15 is prepared using the formulation E disclosed in Table 5. 1.68 grams of risperidone and 2.52 grams of PLGA were dissolved in 10 grams of dichloromethane (DCM) with magnetic agitation at room temperature to produce an API solution (as a first mixture). 5.29 grams of DCM were added in 302.4 grams of 0.1% PVA solution to produce a first solution. Then, the first solution was filtrated via the 0.2 μm filter and cooled to 25° C. The mixer 110 of the mixing system 100 was speeded up to a rotor speed of 5,000 rpm and the first solution was conducted into the in-line recirculating mixing system 100 to remove the air from the conduit 112. The organic phase was injected into the conduit 112 of the in-line recirculating mixing system 100. The API solution and the continuous phase were mixed in the conduit 112 before entering into the mixer 110 to form a second mixture. The second mixture was entered into the mixer 110 and circulated in the in-line recirculating mixing system 100 until the microparticles of the active ingredient(s) were formed, wherein the sizes of microparticles were reduced in the mixer 110, and the microparticles were hardened by the solvent extraction in the conduit 112. The dichloromethane was further evaporated using a mechanical stirrer for 180 minutes at 25° C. Next, the microparticles were collected by centrifugation at 3,000 g for 15 minutes, and washed for three times to remove the PVA solution. Mannitol solution was filtrated with 0.2 μm filter. Microparticles were resuspended by adding water, and mannitol solution was added into the microparticles suspension. The microparticles were stored at −80° C. until lyophilization.

TABLE 5
Conditions for manufacturing microparticles of two-week depot
of risperidone

| | | | | | Clearance | |
|---|---|---|---|---|---|---|
| | | Rotor | Rotor | Tip | between | Shear |
| | | speed | diameter | speed | rotor and | rate |
| Batch | Formulation | (rpm) | (mm) | (m/s) | stator (mm) | (1/s) |
| 14 | E | 2,800 | 18 | 2.6 | 0.5 | 0.005 |
| 15 | E | 5,000 | 18 | 4.7 | 0.5 | 0.009 |
| 16 | E | 5,000 | 18 | 4.7 | 0.5 | 0.009 |

### Example 2 Characterization of the Microparticles of Example 1.2

The microparticles of batches 1 to 16 in example 1.2 were analyzed, in which the particle diameter distribution was determined by use of a dynamic light scattering particle size distribution analyzer, and the yield was also determined; the results are summarized in Table 6. Particle size distribution D10, D50 and D90 are known as the value of the particle size distribution. Particle size distribution D10 is the value of the particle diameter at 10% of the particles size distribution. Particle size distribution D50 is the medium value of the particle size distribution. Particle size distribution D90 is the value of the particle diameter at 90% of the particles size distribution. For microparticles of batches 1 to 16, the shear rates were in the range of 0.005-0.136/s, whereas the particle size distribution for these microparticles was about 4.4 μm to 341.2 μm. The yields were in the range of 27.9% to 87.0%.

Compared with the particles of batches 1 to 13, the particle size distributions of batches 14 to 16 were respectively 79.5 μm to 341.2 μm (batch 14), 11.7 μm to 156.6 μm (batch 15), and 34.5 μm to 217.1 μm (batch 16). The results indicated that the low rotor speed or shear rate would result in microparticles with a broader particle size distribution, such result is not satisfactory. Further, manufacturing processes of the present invention effectively reduce the size distribution of the microparticles.

Due to the narrow particle size distribution, the present method thus eliminates the need of sieving, as generally required in the prior method, thereby leads to a further advantage that potential loss of microparticles to sieving is substantially reduced, which in turn increases the overall yield.

TABLE 6
Characteristics of microparticles of example 1.2

| Batch | D10 (um) | D50 (um) | D90 (um) | Yield |
|---|---|---|---|---|
| 1 | 5.36 | 8.8 | 15.7 | 74.7% |
| 2 | 4.4 | 8.4 | 21.9 | 54.8% |
| 3 | 5.4 | 10.1 | 19.6 | 71.6% |
| 4 | 8.3 | 15.7 | 38.2 | 27.9% |
| 5 | 5.0 | 9.1 | 16.1 | 49.1% |
| 6 | 8.7 | 17.0 | 43.9 | 29.6% |
| 7 | 8.5 | 16.6 | 66.9 | 31.9% |
| 8 | 4.8 | 9.8 | 17.9 | 87.0% |
| 9 | 5.4 | 10.6 | 20.5 | 87.0% |
| 10 | 6.3 | 12.8 | 28.3 | 80.0% |
| 11 | 10.5 | 20.3 | 37.6 | 46.9% |
| 12 | 8.1 | 15.8 | 42.9 | 55.0% |
| 13 | 7.0 | 12.6 | 22.3 | 39.8% |
| 14 | 79.5 | 200.9 | 341.2 | 60.0% |
| 15 | 11.7 | 105.7 | 156.6 | 52.1% |
| 16 | 34.5 | 120.5 | 217.1 | 51.2% |

The condition for manufacturing batch 5 as disclosed in example 1.2 were repeated in other 7 batches, and the results were summarized in Table 7.

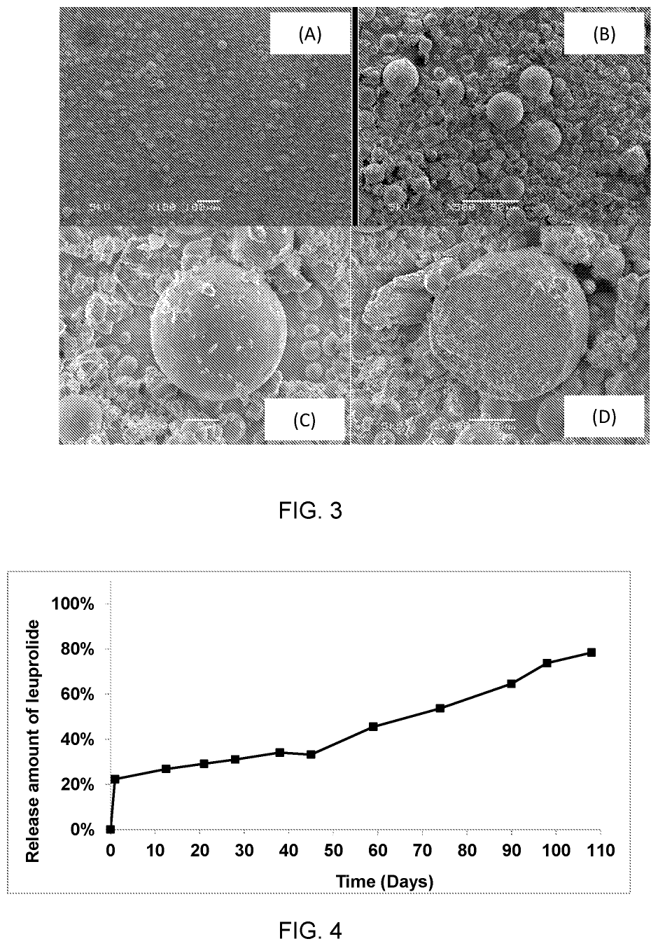

As evident from the data in Table 7, the yield was optimized to about 60%. The microparticles in each batch appeared to be very fine and fairly spherical; with each particles being about 5 to 20 μm in diameter (see the SEM photographs of FIG. 3).

TABLE 7
Characteristics of microparticles of Batch 5
of example 1.2

| Batch | D10 (um) | D50 (um) | D90 (um) | Yield |
|---|---|---|---|---|
| 5-1 | 6.5 | 10.5 | 17.5 | 60.7% |
| 5-2 | 5.6 | 10.1 | 18.9 | 60.2% |
| 5-3 | 5.4 | 9.6 | 18.1 | 64.8% |
| 5-4 | 6.1 | 9.9 | 15.7 | 63.0% |
| 5-5 | 5.9 | 10.7 | 19.5 | 58.2% |
| 5-6 | 6.2 | 10.2 | 16.3 | 59.9% |
| 5-7 | 5.7 | 9.8 | 16.8 | 60.7% |

Taken together, the data from Tables 6 and 7 confirms that the present process is capable of producing microparticles with desirable size in a relatively high yield, in which the distribution in the particle size is narrow.

In addition, the microparticles of batch 5 were also subject to extended release test, where the amounts of leuprolide released from the microparticles were measured over 3-months period of time, and the result is illustrated in FIG. 4. As illustrated, leuprolide continued to release from the microparticles of batch 5 at a steady rate for at least 110 days. Such finding is a proof that present method is capable of producing microparticles with high drug load, which allows continued release of the drug for at least 3-months.

### Example 3 Comparative Examples

In these examples, the microparticles were produced by the conventional batch-type process, and were subject to comparison with those produced by the continuous circulating process of the present invention.

Batches A to C were manufactured by use of formulation B (3-month depot of leuprolide acetate) disclosed in Table 1. Briefly, the leuprolide acetate microparticles of batches A to C were prepared in a batch type mixer using the parameters provided in Table 8. A typical protocol for preparing batch C is described as follows. Briefly, 0.4 grams of leuprolide acetate were dissolved in 0.5 grams of water with magnetic agitation at 40-50° C. to produce an API solution. 2.8 grams of PLA were dissolved in 4.9 grams of dichloromethane (DCM) in the ultrasonic bath, followed by stirring to produce a polymer solution. The API solution was then added into the polymer solution, and emulsified using the homogenizer at the speed of 7,000 rpm for 5 minutes at 15° C., to produce a first mixture. The first mixture was then added into 720 mL 0.5% PVA solution and formed a mixture containing microparticles at a rotor speed of 14,000 rpm at 15° C. using the batch type mixer (i.e. Chemist BOM-300D). DCM was then evaporated using a mechanical stirrer for 180 minutes at room temperature. Next, the microparticles were collected by centrifugation at 2,000 g for 5 minutes, washed with water to remove the PVA solution. Then, mannitol solution was added into the microparticles suspension and was stored at −80° C. until lyophilization. The particle diameter distributions and yields were summarized in Table 9.

TABLE 8
Conditions for manufacturing microparticles in batch type mixer
via batch-type process

| | | | | | Clearance | |
|---|---|---|---|---|---|---|
| | | Rotor | Rotor | Tip | between | Shear |
| | | speed | diameter | speed | rotor and | rate |
| Batch | Formulation | (rpm) | (mm) | (m/s) | stator (mm) | (1/s) |
| A | B | 10,000 | 20 | 10.5 | 0.25 | 0.042 |
| B | B | 14,000 | 20 | 14.7 | 0.25 | 0.059 |
| C | B | 14,000 | 20 | 14.7 | 0.25 | 0.059 |

TABLE 9
Characteristics of microparticles of comparable
example A

| Batch | D10 (um) | D50 (um) | D90 (um) | Yield |
|---|---|---|---|---|
| A | 10.5 | 30.2 | 94.2 | N/A |
| B | 9.4 | 27.3 | 99.0 | 20.1% |
| C | 11.2 | 27.7 | 95.4 | 27.4% |

As to the microparticles of batches A to C, the shear rates were in the range of 0.042-0.059/s and the particle sizes ranged from 9.4 μm to 99.0 μm. The yields were in the range of 20.1% to 27.4%. These data indicated that microparticles prepared by such conventional discontinuous preparation had a broader particle size distribution and a medium to low yield rate.

In conclusion, the experimental data provided herein established that the present process is capable of producing microparticles with desirable size in a yield comparable to that of a batch-type process, even in similar sheer rate, in which the distribution in the particle size is narrow

It will be understood that the above description of embodiments is given by way of example only and that various modifications may be made by those with is ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A method for producing microparticles of an active ingredient via an in-line recirculating mixing system, wherein the in-line recirculating mixing system comprises a mixer and a conduit coupling to the mixer, the method comprises the steps of:
(a) forming a continuous phase of a medium in the in-line recirculating mixing system;
(b) allowing the continuous phase of the medium of the step (a) to come into contact with a first mixture of the active ingredient, a polymer and a solvent at a site in the conduit, thereby forming a second mixture, in the conduit; and
(c) allowing the second mixture of the step (b) to enter the mixer and circulate in the in-line recirculating mixing system until the microparticles of the active ingredient are formed.

2. The method of claim 1, wherein the medium of the step (a) is any of, silicon oil, sorbitan monooleate, polysorbate 20, polysorbate 40, polysorbate 60, polysorbate 65, polysorbate 80, vegetable oil, paraffine oil, polyvinyl alcohol (PVA), polyvinyl pyrrolidone (PVP), carboxyvinyl polymer (CVP), polyvinyl methyl ether (PVME), hydroxyethyl celluloses and poly(sodium acrylate) (PA), fatty acids, sodium lauryl sulfate or alpha olefin sulfonate or equivalent.

3. The method of claim 2, wherein the medium is polyvinyl alcohol.

4. The method of claim 1, wherein in the step (b), the first mixture and the medium come into contact in a volume ratio between 1:20 to 1:1,200.

5. The method of claim 1, wherein the first mixture of the step (b) is prepared by mixing an aqueous solution of the active ingredient and a non-aqueous solution of the polymer and the solvent at a temperature of 4 to 40° C. and a speed at least 7,000 rpm.

6. The method of claim 1, wherein the active ingredient is selected from the group consisting of, a physiologically active peptide, an antitumor agent, an antibiotic, an anti-pyretic agent, an analgesic, an anti-inflammatory agent, an anti-tussive expectorant, a sedative, a muscle relaxant, an anti-epileptic, an anti-ulcer agent, an anti-depressant, an anti-allergic agent, a cardiotonic, an anti-arrhythmic agent, a vasodilator, a hypotensive diuretic, an anti-diabetic, an anti-hyperlipidemic agent, an anti-coagulant, a hemolytic, an anti-tuberculosis agent, a hormone, a narcotic antagonist, a bone resorption suppressor, an osteogenesis promoter and an angiogenesis inhibitor.

7. The method of claim 1, wherein the active ingredient is a physiologically active peptide selected from the group consisting of, growth hormone releasing peptide (GHRP), luteinizing hormone-releasing hormone (LHRH), bombesin, gastrin releasing peptide (GRP), calcitonin, bradykinin, galanin, melanocyte stimulating hormone (MSH), growth hormone releasing factor (GRF), amylin, tachykinins, secretin, parathyroid hormone (PTH), enkephalin, endothelin, calcitonin gene releasing peptide (CGRP), neuromedins, parathyroid hormone related protein (PTHrP), glucagon, neurotensin peptide YY (PYY), glucagon-like peptide-1 (GLP1), liraglutide, exenatide, lixisenatide, albiglutide, dulaglutide, taspoglutide, semaglutide, vasoactive intestinal peptide (VIP), pituitary adenylate cyclase activating peptide (PACAP), motilin, substance P, neuropeptide Y (NPY), thyroid stimulating hormone (TSH), insulin, somatostatin, somatostatin derivative, growth hormones, prolactin, adrenocorticotropic hormone (ACTH), ACTH derivatives, thyrotropin-releasing hormone and salts and derivatives thereof, luteinizing hormone (LH), follicle-stimulating hormone (FSH), vasopressin, vasopressin derivative, oxytocin, calcitonin, gastrin, pancreozymin, cholecystokinin, angiotensin, human placental lactogen, human chorionic gonadotropin (HCG), enkephalin, enkephalin derivatives, endorphin, kyotorphin, interferons, interleukins, tuftsin, thymopoietin, thymosin, thymostimulin, thymic humoral factor (THF), blood thymic factor (FTS) and derivative thereof, other thymic factors, tumor necrosis factor (TNF), colony-stimulating factors, dynorphin, caerulein, bradykinin, urokinase, asparaginase, kallikrein, insulin-like growth factors, nerve growth factor (NGF), cell growth factors, bone morphogenic factor (BMP), nerve nutrition factors, blood coagulation factors VIII and IX, lysozyme chloride, polymixin B, colistin, gramicidin, bacitracin, erythropoietin (EPO), thrombopoietin (TPO) and endothelin-antagonistic peptides.

8. The method of claim 1, wherein the polymer of the step (b) is selected from the group consisting of, polyester, polylactide, polyglycolide, poly(d,l-lactide-co-glycolide), polycaprolactone, polydioxannone, polycarbonate, polyhydroxybutyrate, polyalkyene oxalate, polyanhydride, polyamide, polyesteramide, polyurethane, polyacetal, polyketal, polyorthocarbonate, polyphosphazene, polyhydroxyvalerate, polyalkylene succinate, poly(malic acid), poly(amino acids), chitin, chitosan, gelatin, polyorthoester, polyethylene-polypropylene glycol copolymer, block polymer of polylactides-glycolides with polyethyleneglycol, terpolymer, block copolymer, branched copolymer, polyorthoester, polyanhydride, polyhydroxybutyric acid, polycaprolactone, polyalkylcarbonate, a copolymer or a simple mixture of two or more polymer groups, a copolymer of one of the above-mentioned polymers and polyethylenglycol (PEG), a polymer-sugar complex where a sugar is coupled with one of the above-mentioned polymers or the copolymers, and a combination thereof.

9. The method of claim 1, wherein the solvent of the step (b) is selected from the group consisting of, dichloromethane (DCM), N-methyl-2-pyrrolidone (NMP), aliphatic hydrocarbons, methane (CH4), ethane (C2H6), propane (C3H8), butane (C4H10), pentane (C5H12), hexane (C6H14), heptane (C7H16), octane (C8H18), acetone, acetic acid, chloroform, ethyl acetate, ethyl formate, methyl ketone, ethyl ketone, methyl isobutyl ketone, petroleum ether, 2-pyrrolidone, propylene carbonate, ethylene carbonate, dimethyl carbonate, 2-ethyoxylyl acetate, methyl acetate, ethyl lactate, ethyl butyrate, diethyl malonate, diethyl glutonate, tributyl citrate, diethyl succinate, tributyrin, isopropyl myristate, dimethyl adipate, dimethyl succinate, dimethyl oxalate, dimethyl citrate, triethyl citrate, acetyl tributyl citrate, glyceryl triacetate, methyl ethyl ketone, solketal, glycerol formal, glycofurol, dimethylformamide, dimethylacetamide, dimethylsulfoxide (DMSO), dimethylsulfone, tetrahydrofuran, epsilon-caprolactone, butyrolactone, capro lactam, N,N-dimethyl-m-toluamide, I-dodecylazacycloheptan-2-one, benzyl alcohol, benzyl benzoate and triaetin.

10. The method of claim 8, wherein in the step (b), the polymer is polylactic acid and the solvent is dichloromethane.

11. The method of claim 8, wherein in the step (b), the polymer is poly(d,l-lactide-co-glycolide) and the solvent is dichloromethane.

12. The method of claim 1, wherein in the step (c), the second mixture is circulated in the in-line recirculating mixing system until the microparticles are rigid without forming aggregates.

13. The method of claim 1, wherein in the step (c), the second mixture is subject to a shear rate of 0.010/s to 0.3/s and a temperature of 4 to 40° C. in the mixer.

14. The method of claim 1, wherein in the step (c), the second mixture is circulated in the in-line recirculating mixing system at a constant flow rate of 1 mL/min to 3000 L/min.
